SEQUENCE LISTING (14 sequences)

SEQ ID NO: 1 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 315 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 4 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 8 - protein, 95 aa, Homo sapiens
DVVMTQSPAFLSVTPGEKVTITCQASEGIGNYLYWYQQKPDQAPKLLIKYASQSISGVPSRFSGSGSGTDFTFTISSLEAEDAATYYCQQGNKHP

SEQ ID NO: 9 - protein, 12 aa, Homo sapiens
WTFGQGTKVEIK

SEQ ID NO: 10 - protein, 12 aa, Homo sapiens
YTFGQGTKLEIK

SEQ ID NO: 11 - protein, 12 aa, Homo sapiens
FTFGPGTKVDIK

SEQ ID NO: 12 - protein, 107 aa, Homo sapiens
DVVMTQSPAFLSVTPGEKVTITCQASEGIGNYLYWYQQKPDQAPKLLIKYASQSISGVPSRFSGSGSGTDFTFTISSLEAEDAATYYCQQGNKHPWTFGQGTKVEIK

SEQ ID NO: 13 - protein, 107 aa, Homo sapiens
DVVMTQSPAFLSVTPGEKVTITCQASEGIGNYLYWYQQKPDQAPKLLIKYASQSISGVPSRFSGSGSGTDFTFTISSLEAEDAATYYCQQGNKHPYTFGQGTKLEIK

SEQ ID NO: 14 - protein, 107 aa, Homo sapiens
DVVMTQSPAFLSVTPGEKVTITCQASEGIGNYLYWYQQKPDQAPKLLIKYASQSISGVPSRFSGSGSGTDFTFTISSLEAEDAATYYCQQGNKHPFTFGPGTKVDIK

(19) United States
(12) Patent Application Publication
(10) Pub. No.: US 2012/0020952 A1
(43) Pub. Date: Jan. 26, 2012

(54) METHODS FOR PRODUCING MIXTURES OF ANTIBODIES

(75) Inventors: Paul Parren, Odijk (NL); Joost J. Neijssen, Werkhoven (NL); Aran Frank Labrijn, Amsterdam (NL); Janine Schuurman, Diemen (NL); Tom Vink, Alphen aan den Rijn (NL); Jan Van De Winkel, Zeist (NL); Stefan Loverix, Ternat (BE); Ignace Lasters, Antwerpen (BE)

(73) Assignee: GENMAB A/S, Copenhagen K (DK)

(21) Appl. No.: 13/146,297

(22) PCT Filed: Jan. 26, 2010
(86) PCT No.: PCT/EP2010/050822

§ 371 Date: Oct. 7, 2011

(30) Foreign Application Priority Data
Jan. 26, 2009 (DK) PA 2009 00118

(51) Int. Cl. A61K 31/7088; C07K 16/00; C12N 5/10; C12P 21/00; C12P 21/02

(52) U.S. Cl. 424/130.1; 435/69.6; 435/326; 530/387.1; 530/387.3

(57) ABSTRACT

The invention relates to a method for producing a mixture comprising two or more different antibodies in a single recombinant host cell. In one embodiment, a mixture of different monovalent antibodies is produced. In another embodiment, a mixture of monovalent and bivalent antibodies is produced. The invention also relates to mixtures of antibodies obtainable by the method of the invention and to light chain sequences that are particularly useful in the method of the invention.

### FIELD OF THE INVENTION

The invention provides methods for producing mixtures of antibodies, mixtures obtainable by the methods of the invention and uses of such mixtures, in particular use in the treatment of cancer. The invention also relates to light chains that are particularly useful in the method of the invention.

### BACKGROUND OF THE INVENTION

A number of human diseases are today treated by therapeutic monoclonal antibodies, for example humanized or fully human monoclonal antibodies. However, some diseases are not treated sufficiently effectively by a monoclonal antibody or the treatment looses effect over time with application of monoclonal antibodies, for example due to down-regulation of the target or a switch to a distinct pathogenic pathway. Therefore, an alternative could be treatment with polyclonal antibodies or mixtures of antibodies. Such mixtures of antibodies could comprise two or more antibodies directed against different epitopes on the same target, or alternatively a mixture of antibodies directed against different targets.

U.S. Pat. No. 7,262,028 describes a method for the production of bivalent antibodies or mixtures of bivalent antibodies from a single host cell clone by expression of one light chain and different heavy chains. The invention disclosed in U.S. Pat. No. 7,262,028 provides a method for producing a combination of antibodies which can be screened for the usefulness in a number of applications.

The desired characteristics of therapeutic antibodies may vary according to the specific condition to be treated. For some indications, only antigen binding is required, for instance where the therapeutic effect of the antibody is to block interaction between the antigen and one or more specific molecules otherwise capable of binding to the antigen. For other indications, further antibody-mediated effects may also be required, such as the ability to induce complement activation, to bind Fc receptors, etc. For such use, other parts of the antibody molecule than the antigen binding part, such as the Fc region, may be important. Some full-length antibodies may exhibit agonistic effects (which may be considered to be undesirable, in particular for cancer therapy) upon binding to the target antigen. In some instances, this effect may be attributed to “cross-linking” by bivalent antibodies, which in turn promotes target dimerization, which may lead to activation, especially when the target is a receptor. In the case of soluble antigens, bivalent targeting may form undesirable immune complexes. For some therapeutic indications, monovalent antibodies may thus be preferable.

Examples of monovalent antibodies include Fab fragments, scFv antibodies and nanobodies. Another type of monovalent antibodies (UniBody® molecules), comprising one heavy and one light chain, has been described in WO2007/059782, WO/2008/145137, WO/2008/145138, WO/2008/145139 and WO/2008/145140. In these molecules, the sequences of the heavy chain have been modified so that no inter-heavy chain bonds, and thus no bivalent antibodies, are formed. UniBody® molecules are characterized by favorable pharmacokinetics as compared to Fab fragments.

There is a need for improved antibody-based therapy wherein the advantages of the use of polyclonal antibodies or antibody mixtures are combined with the advantages of monovalent antigen binding. The present invention provides methods for producing mixtures of monovalent antibodies or mixtures of monovalent and bivalent antibodies.

### SUMMARY OF THE INVENTION

In a first main aspect, the invention relates to a method for producing a mixture comprising two or more different antibodies in a single recombinant host cell, comprising expressing in said host cell:

a) at least one nucleic acid construct encoding a common light chain, and

b) two or more nucleic acid constructs encoding a heavy chain, said two or more nucleic acid constructs comprising
b1) two or more nucleic acid constructs encoding two or more different first heavy chains, wherein the amino acid sequence of each of the constant regions of the first heavy chains has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being,
or
b2) at least one nucleic acid construct encoding a first heavy chain, wherein the amino acid sequence of the constant region has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being, and
at least one nucleic acid construct encoding a second heavy chain, which is capable of forming disulphide bonds or covalent or stable non-covalent inter-heavy chain bonds with an identical CH region in the presence of IVIG or when administered to a mammal or human being,
   wherein each of the heavy chains is capable of pairing with the light chain to form a functional antibody.

Thus, the sequences of the first heavy chains have been modified so that no stable inter-heavy chain bonds are formed, and thus the resulting antibodies are monovalent (see e.g. WO2007/059782).

Accordingly, in the case of alternative b1, a mixture of different monovalent antibodies is produced in the same cell. Such a mixture can e.g. be used in the treatment of diseases, in particular diseases where monoclonal monovalent antibodies and/or polyclonal bivalent antibodies are not optimally effective, as explained above.

In the case of alternative b2, a mixture of monovalent and bivalent antibodies is produced in the same cell. Such a mixture can e.g. be used in the treatment of diseases, such as cancer. In a particularly interesting embodiment, the monovalent antibody inhibits cell proliferation by antagonistic binding to or blocking of a target protein and the bivalent antibody binds another target antigen, for instance on the same target cell, and recruits effector functions for target cell killing.

In a further main aspect, the invention relates to a composition, such as a pharmaceutical composition, comprising a mixture of antibodies obtained or obtainable by the method of the invention.

In an even further aspect, the invention provides a recombinant host cell suitable for producing mixtures of antibodies according to the method above.

The invention also provides the use of the compositions according to the invention above for treatment of diseases.

The present invention also discloses a common light chain that is particularly suitable for use in the present invention, because it can replace light chains of various different antigen-specific antibodies without loss of specificity. Such a light chain may more generally be used for antibody products containing applications wherein one light chain is to be combined with multiple heavy chains, such as in recombinant polyclonal or bispecific antibodies.

Thus, in a further aspect, the invention relates to a recombinant antibody comprising a heavy chain and a light chain, wherein the light chain comprises the sequence as set forth in SEQ ID NO.8.

### BRIEF DESCRIPTION OF THE FIGURES AND THE SEQUENCE LISTING

FIG. 1. Binding of monovalent antibodies, present in the cell culture supernatant of transfected HEK-293F cells, to soluble His-tagged CD38 was measured in an ELISA. “Monovalent Uni-005” indicates supernatant of HEK-293F cells transfected with a monovalent Uni-005 (anti-CD38) construct; “Monovalent combination” indicates a supernatant of cells expressing a combination of the heavy chains of Uni-7D8 (anti-CD20) and Uni-005 with the light chain of the anti-CD38 antibody 005.

FIG. 2. Binding of monovalent antibodies, present in the cell culture supernatant of transfected HEK-293F cells, to an anti-idiotype antibody against HuMab-7D8, which also binds Uni-7D8, was measured in an ELISA. “Monovalent Uni-7D8” indicates a supernatant of HEK-293F cells transfected with a Uni-7D8 construct; “Monovalent combination” indicates a supernatant of cells expressing a combination of the heavy chains of Uni-7D8 and Uni-CD38 with the light chain of anti-CD38 antibody 005.

FIG. 3: Screening of human Kappa light chain germline library for binding to various heavy chains to identify common light chains. Supernatants of transient transfected HEK-293F cells expressing a hinge-modified (F273T, Y275E) heavy chain with a variable domain specific for EGFr (A), c-Met (B) or Her2 (C) and a single germline kappa light chain from the library were screened for binding in an ELISA using recombinant soluble antigen as coat. Each dot represents a unique heavy and light chain combination and binding (OD405) and expression (μg/mL after 1:20 dilution) is shown. Combinations of heavy and light chains that form functional binding antibodies are marked with 1, 2 and 3. (1′, 2′ and 3′ are duplicates of 1, 2 and 3 in the assay). The original kappa light chain accompanying the particular heavy chain was included in each experiment as a positive control (rectangles)

FIG. 4: Confirmation of common light chains by co-expression and binding ELISA to recombinant target. Supernatants of transient transfected HEK-293F cells co-expressing three hinge-modified (F273T, Y275E) heavy chains with a variable domain specific for EGFR, c-Met and Her2, respectively, and a single common kappa light chain germline sequence that was identified in the primary screen (1, 2 or 3 in FIG. 3) were tested for binding in an ELISA using recombinant soluble antigen (EGFR (A), c-Met (B) or Her2 (C), respectively) as coat. The original kappa light chain accompanying the particular heavy chain was included in each experiment as a positive control.

FIG. 5: Determination of monovalency of antibodies by cross-linking ELISA. Supernatants of transient transfected HEK-293F cells co-expressing three hinge-modified (F273T, Y275E) heavy chains with a variable domain specific for EGFR and c-Met, respectively, and a single common kappa light chain germline sequence that was identified in the primary screen (1, 2 and 3 in FIG. 3) were tested for monovalency in a crosslink ELISA using recombinant soluble antigen (EGFr (A) and c-Met (B)) as coat and the same antigen conjugated to biotin as detection for bivalent molecules. IgG1 antibodies against EGFR and c-Met were used as positive control in the assay and a control batch of monovalent antibodies against EGFR and c-Met as negative controls.

SEQ ID NO: 1: Amino acid sequence of the wild type constant domain of the heavy chain (CH) of human IgG4 (accession number P01861). Sequences in italics represent the CH1 region, highlighted sequences represent the hinge region, regular sequences represent the CH2 region and underlined sequences represent the CH3 region.

SEQ ID NO: 2: Amino acid sequence of the mutant constant region of the heavy chain (CH) of human IgG4 in which the hinge region is deleted.

SEQ ID NO: 3: Amino acid sequence of the constant domain of the human lambda light chain (CL) (accession number S25751).

SEQ ID NO: 4: Amino acid sequence of the constant domain of the human kappa light chain (CL) (accession number P01834).

SEQ ID NO: 5: Amino acid sequence of the constant domain of the heavy chain (CH) of human IgG1 (accession number P01857). Sequences in italics represent the CH1 region, highlighted sequences represent the hinge region, regular sequences represent the CH2 region and underlined sequences represent the CH3 region.

SEQ ID NO: 6: Amino acid sequence of the constant domain of the heavy chain (CH) of human IgG2 (accession number P01859). Sequences in italics represent the CH1 region, highlighted sequences represent the hinge region, regular sequences represent the CH2 region and underlined sequences represent the CH3 region.

SEQ ID NO: 7: Amino acid sequence of the constant domain of the heavy chain (CH) of human IgG3 (accession number P01860). Sequences in italics represent the CH1 region, highlighted sequences represent the hinge region, regular sequences represent the CH2 region and underlined sequences represent the CH3 region.

SEQ ID NO: 8: Amino acid sequence of V-segment VKVI-2-1-(1)-A14 (IGKV6D-41*01).

SEQ ID NO: 9: Amino acid sequence of JK-segment JK1 (IGKJ1*01)

SEQ ID NO: 10: Amino acid sequence of JK-segment JK2 (IGKJ2*01)

SEQ ID NO: 11: Amino acid sequence of JK-segment JK3 (IGKJ3*01)

SEQ ID NO: 12: Amino acid sequence of common light chain 1

SEQ ID NO: 13: Amino acid sequence of common light chain 2

SEQ ID NO: 14: Amino acid sequence of common light chain 3

### DETAILED DESCRIPTION OF THE INVENTION

### Definitions

Unless specified otherwise, the term “antibody” as referred to herein includes whole antibody molecules, antigen binding fragments, monovalent antibodies, and single chains thereof. Antibody molecules belong to a family of plasma proteins called immunoglobulins, whose basic building block, the immunoglobulin fold or domain, is used in various forms in many molecules of the immune system and other biological recognition systems. Native antibodies and immunoglobulins are usually heterotetrameric glycoproteins of about 150,000 Dalton, composed of two identical light (L) chains and two identical heavy (H) chains. Each heavy and light chain may also have regularly spaced intrachain disulfide bridges. Each light chain is comprised of a light chain variable region (abbreviated herein as VL) and a light chain constant region (abbreviated herein as CL). Each heavy chain is comprised of a heavy chain variable region (VH) and a heavy chain constant region (CH) consisting of three homologous domains (CH1, CH2 and CH3) and the hinge region. The constant domain of the light chain is aligned with the first constant domain (CH1) of the heavy chain, and the light chain variable domain is aligned with the variable domain of the heavy chain forming what is known as the “Fab”, for antigen binding fragment. CH1 and CH2 of the heavy chain are separated from each other by the hinge region, which flexibility allows the Fab “arms” of the antibody molecule to move to some degree from the Fc part. The hinge region normally comprises one or more cysteine residues, which are capable of forming disulphide bridges with the cysteine residues of the hinge region of the other heavy chain within one antibody molecule.

The variable regions of the heavy and light chains form the binding domain that interacts with an antigen. Antibodies interact with target antigens primarily through amino acid residues that are located in the six heavy and light chain complementarity determining regions (CDRs). For this reason, the amino acid sequences within CDRs are more diverse between individual antibodies than sequences outside of CDRs. The constant regions of the antibodies may mediate the binding of the immunoglobulin to host tissues or factors, including various cells of the immune system (for instance effector cells) and the first component of the classical complement system (C1q).

Depending on the amino acid sequences of the constant domain of their heavy chains, immunoglobulins can be classified in at least five (5) major classes of immunoglobulins: IgA, IgD, IgE, IgG and IgM. Several of these classes may be further divided into subclasses (isotypes), for instance IgG1, IgG2, IgG3 and IgG4; IgA1 and IgA2. The genes encoding the heavy chain constant domains of immunoglobulins are called alpha (α) for IgA, delta (δ) for IgD, epsilon (ε) for IgE, gamma (γ) for IgG and mu (μ) for IgM. The IgG subclasses are encoded by different genes: γ1 for IgG1, γ2 for IgG2, γ3 for IgG3 and γ4 for IgG4. The light chains of antibodies are assigned to one of two clearly distinct types, called kappa (κ) and lambda (λ), based on the amino sequences of their constant domain. The three-dimensional structure of different classes of immunoglobulins is well known and can be divided in subunits. Comparison within the IgG heavy chain defines the CH1, CH2 and CH3 homology regions. These regions are indicated for the different IgG isotypes in the sequence listing herein. Comparisons between homology regions of each of the four IgG subclasses reveals >95% sequence identity (Jefferis, R. 1990. F. Shakib, ed. Pergamon Press, Oxford, p. 15). Distinct allotypes of immunoglobulins exist within the human population, such as G1m(a), G1m(x), G1m(f) and G1m(z) for the IgG1 heavy chain and Km1, Km1,2 and Km3 for the kappa light chain. These allotypes differ at distinct amino acids in their constant regions. The sequence between the CH1 and CH2 domains is referred to as the hinge region because it allows molecular flexibility. The CH3 domains of the two heavy chains within one antibody are paired and the non-covalent interactions are sufficient for the IgG molecule to maintain its structural integrity following reduction of the inter-heavy chain disulphide bridges under mild conditions. CH3 domain pairing is compact and similar to pairing in the Fab, with a nearly exact dyad between the two domains (Saphire, et al., 2002. J Mol Biol 319:9). This is in contrast to the CH2 domains, which do not associate closely and their contact is primarily mediated by the two carbohydrate chains attached to the Asn297 residues (Saphire, et al., 2002. J Mol Biol 319:9). The characteristic IgG structure in which two heavy-light chain heterodimers are linked is thus maintained by the inter-heavy chain disulphide bridges of the hinge region and the non-covalent interactions of the CH3 domains.

In the context of the present invention a common light chain” refers to light chains which may be identical or have amino-acid sequence differences. Common light chains may comprise mutations which do not alter the specificity of the antibody when combined with the same heavy chain without departing from the scope of the present invention. It is, for instance, possible within the scope of the definition of common light chains as used herein, to prepare or find light chains that are not identical but still functionally equivalent, e.g., by introducing and testing conservative amino acid changes or changes of amino acids in regions that do not or only partly contribute to binding specificity when paired with the heavy chain. In an exemplary embodiment, the present invention provides the use of a common light chain, one identical light chain, to combine with different heavy chains to form antibodies with functional antigen-binding domains. The use of one common light chain avoids the formation of heterodimers in which pairing of light and heavy chains results in antigen-binding domains that are not functional or, in other words, which are not capable of binding to the target antigen or antigens.

In the context of the present invention “different heavy chains” means heavy chains which differ in the variable regions. The different heavy chains may have identical or different constant regions.

The term “human antibody”, as used herein, is intended to include antibodies having variable and constant regions derived from human germline immunoglobulin sequences. The human antibodies of the invention may include amino acid residues not encoded by human germline immunoglobulin sequences (for instance mutations introduced by random or site-specific mutagenesis in vitro or by somatic mutation in vivo). However, the term “human antibody”, as used herein, is not intended to include antibodies in which CDR1 or CDR2 sequences derived from the germline of another mammalian species, such as a mouse, or the CDR3 region derived from an antibody from another species than human, such as mouse, have been grafted onto human framework sequences.

The term “K_{D}”, as used herein, refers to the dissociation equilibrium constant of a particular antibody-antigen interaction, in mol (M).

The terms “monoclonal antibody” or “monoclonal antibody composition” as used herein refer to a preparation of antibody molecules of a single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences.

The term “nucleic acid”, “nucleic acid construct” or “nucleic acid molecule”, as used herein, is intended to include DNA molecules and RNA molecules. A nucleic acid molecule may be single-stranded or double-stranded.

As used herein, “specific binding” refers to the binding of an antibody, or antigen-binding fragment thereof, to a predetermined antigen. Typically, the antibody binds to a predetermined antigen with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, when measured for instance using sulfon plasmon resonance on BIAcore or as apparent affinities based on IC50 values in FACS or ELISA, and binds to the predetermined antigen with an affinity corresponding to a K_{D} that is at least ten-fold lower, such as at least 100 fold lower, for instance at least 1,000 fold lower, such as at least 10,000 fold lower, for instance at least 100,000 fold lower than its affinity for binding to a non-specific antigen (e.g., BSA, casein) other than the predetermined antigen or a closely-related antigen. The amount with which the affinity is lower is dependent on the K_{D} of the antibody, so that when the K_{D} of the antibody is very low (that is, the antibody is highly specific), then the amount with which the affinity for the antigen is lower than the affinity for a non-specific antigen may be at least 10,000 fold.

The terms “non-human transgenic animal” refers to a non-human animal having a genome comprising one or more human heavy and/or light chain loci on transgenes or transchromosomes and which is capable of expressing human antibodies. For example, a transgenic mouse can have a human light chain locus on a transgene and either a human heavy chain locus on a transgene or a human heavy chain locus on a transchromosome, such that the mouse produces human antibodies when immunized with an antigen and/or cells expressing an antigen. The human heavy chain transgene can be integrated into the chromosomal DNA of the mouse, as is the case for transgenic, for instance HuMAb™ mice, such as HCo7 or HCo12 mice, or the human heavy chain transgene can be maintained extrachromosomally within a human chromosome fragment, as is the case for the transchromosomal KM-Mouse™ as described in WO 02/43478. Such transgenic and transchromosomal mice are capable of producing multiple isotypes of human antibodies binding to selected antigens (e.g., IgG, IgA and/or IgE) by undergoing V-D-J recombination and isotype switching.

The term “valence of an antibody” means the maximum number of antigenic determinates with which the antibody can react. For example wild type IgG antibodies contain two Fab regions and can bind two molecules of antigen or two identical sites on the same particle, and thus have a valence of two (“bivalent”). The term “monovalent antibody” means in the present context that an antibody molecule at most contains one Fab region and normally is capable of binding a single molecule of the antigen only, and thus is not able to mediate antigen crosslinking.

The term “epitope” means a protein determinant capable of specific binding to an antibody. Epitopes usually consist of chemically active surface groupings, such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents.

The term “host cell” (or “recombinant host cell”), as used herein, is intended to refer to a cell into which a recombinant expression vector has been introduced. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein. The term “host cell” in singular form may also denote a culture of a specific kind of host cell. Expression of the antibodies according to the invention may occur through the use of any host cell capable of expressing recombinant DNA molecules, including bacteria, such as E. coli, Enterobactor, Salmonella, Bacillus, Pseudomonas and Streptomyces, yeasts, such as S. cerevisiae, K. lactis, P. pastoris, Candida and Yarrowia, filamentous fungi, such as Neurospora, Aspergillus oryzae, Aspergillus nidulans and Aspergillus niger, plant cells, such as Arabidopsis, insect cells, such as Spodoptera frugiperda SF-9 and SF-21 cells, mammalian cells, such as Chinese hamster ovary (CHO cells), BHK cells, mouse cells, including SP2/0 and NS-0 myeloma cells, primate cells, such as COS and Vero cells, MDCK cells, BRL 3A cells, hybridomas, tumor cells, immortalized primary cells, human cells, such as W138, HepG2, HeLa, HEK-293, HT1080 or embryonic retina cells, such as PER.C6™ and the like. The choice of the cell inter alia depends on the glycosylation pattern to be obtained.

The term “IVIG” refers to intravenous immunoglobulin as prepared by Sanquin, the Netherlands. In brief, IVIG is prepared from a pool of human plasma of at least 1,000 donors by a modified Cohn ethanol fractionation technique described by Brummelhuis (1983) Acta Pharmac Scand (suppl) 4:91. The preparation is made suitable for intravenous administration by treating the Cohn fraction II at pH 4 in the presence of a trace of pepsin. The material is being provided in lyophilized form. After being dissolved in the specified volume, the product contains about 60 gram protein per liter. The protein fraction contains at least 95% IgG and small amounts of IgA (<2 gram per liter) and IgM and traces of other plasma proteins. The content of the IgG subclasses is comparable to that of normal human plasma: 60% IgG1, 33% IgG2, 3% IgG3 and 3% IgG4. The preparation contains 0.24 mol glucose and 37 mmol sodium per liter.

### FURTHER ASPECTS AND EMBODIMENT OF THE INVENTION

As explained above, in one aspect, the invention relates to an in vitro method for producing a mixture comprising two or more different antibodies in a single recombinant host cell, comprising expressing in said host cell:

a) at least one nucleic acid construct encoding a common light chain, and

b) two or more nucleic acid constructs encoding a heavy chain, said two or more nucleic acid constructs comprising
b1) two or more nucleic acid constructs encoding two or more different first heavy chains, wherein the amino acid sequence of each of the constant regions of the first heavy chains has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being,
or
b2) at least one nucleic acid construct encoding a first heavy chain, wherein the amino acid sequence of the constant region has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being, and
at least one nucleic acid construct encoding a second heavy chain which is capable of forming disulphide bonds or covalent or stable non-covalent inter-heavy chain bonds with an identical CH region in the presence of IVIG or when administered to a mammal or human being,
   wherein each of the heavy chains is capable of pairing with the light chain to form a functional antibody.

Accordingly, in one embodiment, the method of the invention comprises expressing in said host cell:

two or more nucleic acid constructs encoding two or more different first heavy chains, wherein the amino acid sequence of each of the constant regions of the first heavy chains has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being.

The resulting composition thus comprises two or more different monovalent antibodies.

In an alternative embodiment, the method of the invention comprises expressing in said host cell:
at least one nucleic acid construct encoding a first heavy chain, wherein the amino acid sequence of the constant region has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being, and
at least one nucleic acid construct encoding a second heavy chain which is capable of forming disulphide bonds or covalent or stable non-covalent inter-heavy chain bonds with an identical CH region in the presence of IVIG or when administered to a mammal or human being.

The resulting composition thus comprises a mixture of monovalent and bivalent antibodies.

The monovalent antibodies comprised within the mixture of antibodies produced by the method of the invention may in principle be of any isotype, including, but not limited to IgG1, IgG2, IgG3, IgG4, IgA1 and IgA2.

Accordingly, in one embodiment, the monovalent antibodies are derived from IgG1, but have been modified to further reduce intermolecular interactions. Thus, in one embodiment of the method of the invention, said at least one nucleic acid construct encoding a first heavy chain comprises a sequence encoding a CH3 region as set forth in SEQ ID NO: 5, but wherein the CH3 region has been modified so that one or more of the following amino acid substitutions have been made: Arg (R) in position 238 has been replaced by Gln (Q); Asp (D) in position 239 has been replaced by Glu (E); Thr (T) in position 249 has been replaced by Ala (A); Leu (L) in position 251 has been replaced by Ala (A) or Val (V); Phe (F) in position 288 has been replaced by Ala (A) or Leu (L); Tyr (Y) in position 290 has been replaced by Ala (A); Lys (K) in position 292 has been replaced by Arg (R) or Ala (A); Gln (Q) in position 302 has been replaced by Glu (E); and Pro (P) in position 328 has been replaced by Leu (L). In a preferred embodiment, Lys (K) in position 292 has been replaced by Arg (R).

In a further preferred embodiment, said at least one nucleic acid construct encoding a first heavy chain further comprises a sequence encoding the CH1 and/or CH2 regions as set forth in SEQ ID NO: 5.

In case the CH region is of the IgG1 isotype, the constant region may be optionally further modified, because in an IgG1, a free cysteine residue of the light chain could potentially keep the antibody in a bivalent form, even in the absence of cysteines in the hinge region.

Thus, in one embodiment, the constant region of the light chain has been modified so that it does not contain any amino acids capable of forming disulfide bonds or other covalent bonds with an identical constant region in the presence of IVIG or when administered to a mammal or human being. For example, said at least one nucleic acid construct encoding a common light chain comprises a sequence encoding the kappa CL region having the amino acid sequence as set forth in SEQ ID NO: 4, but wherein the sequence has been modified so that the terminal cysteine residue in position 106 has been replaced with another amino acid residue or has been deleted, or said at least one nucleic acid construct encoding a common light chain comprises a sequence encoding the lambda CL region having the amino acid sequence as set forth in SEQ ID NO: 3, but wherein the sequence has been modified so that the cysteine residue in position 104 has been replaced with another amino acid residue or has been deleted.

Alternatively, the constant region of the heavy chain has been modified so that it contains a residue that is capable of forming a disulfide bond or other covalent bond with the light chain. For example, said at least one nucleic acid construct encoding a first heavy chain comprises a sequence encoding a CH1 region as set forth in SEQ ID NO: 5, but wherein the CH1 region has been modified so that Ser (S) in position 14 has been replaced by a cysteine residue.

In a further embodiment, the monovalent antibodies are derived from IgG2, but have been modified to further reduce intermolecular interactions. Thus, in one embodiment of the method of the invention, said at least one nucleic acid construct encoding a first heavy chain comprises a sequence encoding a CH3 region as set forth in SEQ ID NO: 6, but wherein the CH3 region has been modified so that one or more of the of the following amino acid substitutions have been made: Arg (R) in position 234 has been replaced by Gln (Q); Thr (T) in position 245 has been replaced by Ala (A); Leu (L) in position 247 has been replaced by Ala (A) or Val (V); Met (M) in position 276 has been replaced by Val (V); Phe (F) in position 284 has been replaced by Ala (A) or Leu (L); Tyr (Y) in position 286 has been replaced by Ala (A); Lys (K) in position 288 has been replaced by Arg (R) or Ala (A); Gln (Q) in position 298 has been replaced by Glu (E); and Pro (P) in position 324 has been replaced by Leu (L). In a preferred embodiment, Lys (K) in position 288 has been replaced by Arg (R).

In a further preferred embodiment, said at least one nucleic acid construct encoding a first heavy chain further comprises a sequence encoding the CH1 and/or CH2 regions as set forth in SEQ ID NO: 6.

In a further embodiment, the monovalent antibodies are derived from IgG3, but have been modified to further reduce intermolecular interactions. Thus, in one embodiment of the method of the invention, said at least one nucleic acid construct encoding a first heavy chain comprises a sequence encoding a CH3 region as set forth in SEQ ID NO: 7, but wherein the CH3 region has been modified so that one or more of the following amino acid substitutions have been made: Arg (R) in position 285 has been replaced by Gln (Q); Thr (T) in position 296 has been replaced by Ala (A); Leu (L) in position 298 has been replaced by Ala (A) or Val (V); Ser (S) in position 314 has been replaced by Asn (N); Asn (N) in position 322 has been replaced by Lys (K); Met (M) in position 327 has been replaced by Val (V); Phe (F) in position 335 has been replaced by Ala (A) or Leu (L); Tyr (Y) in position 337 has been replaced by Ala (A); Lys (K) in position 339 has been replaced by Arg (R) or Ala (A); Gln (Q) in position 349 has been replaced by Glu (E); Ile (I) in position 352 has been replaced by Val (V); Arg (R) in position 365 has been replaced by His (H); Phe (F) in position 366 has been replaced by Tyr (Y); and Pro (P) in position 375 has been replaced by Leu (L). In a preferred embodiment, Lys (K) in position 339 has been replaced by Arg (R).

In a further preferred embodiment, said at least one nucleic acid construct encoding a first heavy chain further comprises a sequence encoding the CH1 and/or CH2 regions as set forth in SEQ ID NO: 7.

In a particularly interesting aspect of the invention, the monovalent antibodies comprised within the mixture produced by the method of the invention are of the IgG4 isotype. Thus, in a further aspect, the invention relates to a method for producing a mixture comprising two or more different antibodies in a single recombinant host cell, comprising expressing in said host cell

a) at least one nucleic acid construct encoding a common light chain, and

b) two or more nucleic acid constructs encoding a heavy chain, said two or more nucleic acid constructs comprising
b1) two or more nucleic acid constructs encoding two or more different first IgG4 heavy chains, wherein the amino acid sequence of each of the constant regions of the first IgG4 heavy chains has been modified so that the hinge region does not contain any amino acid residues which are capable of forming disulphide bonds or covalent or stable non-covalent inter-heavy chain bonds with an identical CH region in the presence of IVIG or when administered to a mammal or human being,
or
b2) at least one nucleic acid construct encoding a first IgG4 heavy chain, wherein the amino acid sequence of the constant region has been modified so that the hinge region does not contain any amino acid residues which are capable of forming disulphide bonds or covalent or stable non-covalent inter-heavy chain bonds with an identical CH region in the presence of IVIG or when administered to a mammal or human being, and
at least one nucleic acid construct encoding a second heavy chain which is capable of forming disulphide bonds or covalent or stable non-covalent inter-heavy chain bonds with an identical CH region in the presence of IVIG or when administered to a mammal or human being, with the proviso that the second heavy chain is not a wild type IgG4 heavy chain,
   wherein each of the heavy chains is capable of pairing with the light chain to form a functional antibody.

In one embodiment, the monovalent antibodies are derived from IgG4, but have been modified to further reduce intermolecular interactions. Thus, in one embodiment of the method of the invention, said at least one nucleic acid construct encoding a first heavy chain comprises a sequence encoding a CH3 region as set forth in SEQ ID NO: 2, but wherein the CH3 region has been modified so that one or more of the following amino acid substitutions have been made: Tyr (Y) in position 217 has been replaced by Arg (R); Leu (L) in position 219 has been replaced by Asn (N) or Gln (Q); Glu (E) in position 225 has been replaced by Ala (A), Thr (T), Val (V) or Ile (I); Ser (S) in position 232 has been replaced by Arg (R) or Lys (K); Thr (T) in position 234 has been replaced by Ala (A), Arg (R), Lys (K) or Asn (N); Leu (L) in position 236 has been replaced by Ala (A), Val (V), Glu (E), Gly (G), Ser (S) or Thr (T); Lys (K) in position 238 has been replaced by Ala (A), Arg (R) or Thr (T); Asp (D) in position 267 has been replaced by Ala (A), Thr (T) or Ser (S); Phe (F) in position 273 has been replaced by Ala (A), Leu (L), Thr (T), Asp (D), Arg (R), Gln (Q), Lys (K) or Tyr (Y); Tyr (Y) in position 275 has been replaced by Ala (A), Glu (E), Gln (Q), Lys (K) or Phe (F); Arg (R) in position 277 has been replaced by Ala (A), Lys (K) or Glu (E); Thr (T) in position 279 has been replaced by Asp (D), Val (V) or Asn (N). In a preferred embodiment, Leu (L) in position 236 has been replaced by Val (V).

In a further embodiment, said at least one nucleic acid construct encoding a first heavy chain comprises a sequence encoding a CH3 region as set forth in SEQ ID NO: 2.

In a further embodiment, Phe (F) in position 273 has been replaced by Asp (D) and/or Tyr (Y) in position 275 has been replaced by Glu (E).

In a further embodiment, Phe (F) in position 273 has been replaced by Thr (T) and/or Tyr (Y) in position 275 has been replaced by Glu (E).

In a further embodiment, Tyr (Y) in position 275 has been replaced by Ala (A).

In a further embodiment, said at least one nucleic acid construct encoding a first heavy chain further comprises a sequence encoding the CH2 region as set forth in SEQ ID NO: 2, but wherein Thr (T) in position 118 has been replaced by Gln (Q) and/or Met (M) in position 296 has been replaced by Leu (L).

In a further embodiment, said at least one nucleic acid construct encoding a first heavy chain further comprises a sequence encoding the CH2 region as set forth in SEQ ID NO: 2, but wherein one, two or all three of the following substitutions have been made: Met (M) in position 120 has been replaced by Tyr (Y); Ser (S) in position 122 has been replaced by Thr (T); and Thr (T) in position 124 has been replaced by Glu (E).

In a further embodiment, said at least one nucleic acid construct encoding a first heavy chain further comprises a sequence encoding the CH2 region as set forth in SEQ ID NO: 2, but wherein Asn (N) in position 302 has been replaced by Ala (A). In a further embodiment, said at least one nucleic acid construct encoding a first heavy chain further comprises a sequence encoding the CH2 region as set forth in SEQ ID NO: 2, but wherein Asn (N) in position 302 has been replaced by Ala (A) and Thr (T) in position 175 has been replaced by Ala (A) and Glu (E) in position 248 has been replaced by Ala (A).

The modification of the hinge region may be performed in several ways.

In one embodiment, said at least one nucleic acid construct encoding a first heavy chain comprises a sequence encoding a CH region which has been modified such that all cysteine residues in the hinge region have been deleted or substituted with other amino acid residues.

In a further embodiment, the CH region has been modified such that the cysteine residues of the hinge region have been substituted with amino acid residues that have an uncharged polar side chain or a nonpolar side chain.

In a further embodiment, the said at least one nucleic acid construct encoding a first heavy chain comprises a sequence encoding a IgG4 CH region, wherein the amino acids corresponding to amino acids 106 and 109 of the CH sequence of SEQ ID No: 1 have been deleted.

In a further embodiment, said at least one nucleic acid construct encoding a first heavy chain comprises a sequence encoding a IgG4 CH region, wherein one of the amino acid residues corresponding to amino acid residues 106 and 109 of the sequence of SEQ ID No: 1 has been substituted with an amino acid residue different from cysteine, and the other of the amino acid residues corresponding to amino acid residues 106 and 109 of the sequence of SEQ ID No: 1 has been deleted.

In a further embodiment, said at least one nucleic acid construct encoding a first heavy chain comprises a sequence encoding an IgG4 CH region, wherein at least the amino acid residues corresponding to amino acid residues 106 to 109 of the CH sequence of SEQ ID No: 1 has been deleted.

In a further embodiment, said at least one nucleic acid construct encoding a first heavy chain comprises a sequence encoding a IgG4 CH region, wherein at least the amino acid residues corresponding to amino acid residues 99 to 110 of the sequence of SEQ ID No: 1 have been deleted.

In a further embodiment, said at least one nucleic acid construct encoding a first heavy chain comprises a sequence encoding a CH region which, except for any mutations specified in any of the preceding claims, comprises the amino acid sequence of SEQ ID No: 2.

In an even further embodiment, said at least one nucleic acid construct encoding a first heavy chain comprises a sequence encoding an IgG4 CH region, wherein the CH region has been modified such that the entire hinge region has been deleted.

The monovalent antibodies may optionally comprise further modifications. In one embodiment, said at least one nucleic acid construct encoding a first heavy chain comprises a sequence encoding a CH region which has been modified so that it does not comprise any acceptor sites for N-linked glycosylation. Preferably, the NST acceptor site for N-linked glycosylation in the CH2 region has been modified to a sequence selected from the group consisting of: GST, MST, CSE, DSE, DSP, ESP, GSP, HSE, NSE, PSP and SSE.

In a further embodiment of the method of the invention, at least one, at least two, e.g. all of the antibodies in the mixture are human antibodies.

In a further embodiment of the method of the invention, the common light chain comprises the sequence as set forth in SEQ ID NO.8.

In an even further embodiment hereof, the common light chain further comprises a sequence selected from the group consisting of: SEQ ID NO:9, 10 and 11, such as a common light chain comprising a sequence selected from the group consisting of: SEQ ID NO:12, 13 and 14.

In another embodiment of the method of the invention, a mixture of three or more different antibodies, such as a mixture of four or more different antibodies, e.g. a mixture of five or more different antibodies is produced.

In a further embodiment, a mixture of less than twenty different antibodies is produced.

In an even further embodiment, said host cell comprises more than one nucleic acid construct encoding a light chain, preferably wherein each of the heavy chains is capable of pairing with each of the light chain to form a functional antibody.

In an even further embodiment, the method comprises culturing said host cell for at least 20 population doublings.

In an even further embodiment, the method comprises the further step of harvesting the mixture from the cell culture.

In an even further embodiment, the method comprises the further step of purifying the antibody mixture.

In an even further embodiment, said nucleic acid is stably integrated into the genome of the host cell.

In a further aspect, the invention relates to a composition comprising a mixture of antibodies obtainable by the method of the invention. In one embodiment, said composition is use as a medicament.

In an even further aspect, the invention relates to a recombinant host cell suitable for use in the production of a mixture comprising two or more different antibodies, wherein said host cell comprises:

a) at least one nucleic acid construct encoding a common light chain, and

b) two or more nucleic acid constructs encoding a heavy chain, said two or more nucleic acid constructs comprising
b1) two or more nucleic acid constructs encoding two or more different first heavy chains, wherein the amino acid sequence of each of the constant regions of the first heavy chains has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being,
or
b2) at least one nucleic acid construct encoding a first heavy chain, wherein the amino acid sequence of the constant region has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being, and
at least one nucleic acid construct encoding a second heavy chain which is capable of forming disulphide bonds or covalent or stable non-covalent inter-heavy chain bonds with an identical CH region in the presence of IVIG or when administered to a mammal or human being.

In one embodiment, said host cell is a mammalian cell, such as a CHO cell.

### Mixtures of Monovalent and Bivalent Antibodies

As explained above, in one embodiment, the invention relates to a method for producing a mixture comprising two or more different antibodies in a single recombinant host cell, comprising expressing in said host cell:

a) at least one nucleic acid construct encoding a common light chain, and

b) two or more nucleic acid constructs encoding a heavy chain, said two or more nucleic acid constructs comprising
at least one nucleic acid construct encoding a first heavy chain, wherein the amino acid sequence of the constant region has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being, and
at least one nucleic acid construct encoding a second heavy chain which is capable of forming disulphide bonds or covalent or stable non-covalent inter-heavy chain bonds with an identical CH region in the presence of IVIG or when administered to a mammal or human being,
   wherein each of the heavy chains is capable of pairing with the light chain to form a functional antibody.

Thus, in this embodiment, a mixture of monovalent and bivalent antibodies is produced in the same cell. Such a mixture can e.g. be used in the treatment of diseases, such as cancer. In a particularly interesting embodiment, the monovalent antibody inhibits cell proliferation by blocking a target protein and the bivalent antibody binds another target antigen, for instance on the same target cell, and recruits effector functions for target cell killing.

Preferably, the second heavy chain is an IgG1, IgG2, IgG3, IgA or stabilized IgG4 heavy chain.

Most preferably, the second heavy chain is of an isotype, e.g. IgG1, which allows the formation of a bivalent antibody which is capable of activating effector functions, such as ADCC and CDC.

In another embodiment, the second heavy chain is IgG4 based, but has been modified so as to stabilize the IgG4 molecule (i.e. prevent dynamic Fab arm exchange as described in van den Neut Kolfschoten (2007) Science 317:1507). Such a stabilized IgG4 does not activate effector mechanisms, but does crosslink receptors. Stabilized IgG4 antibodies have been described in PCT/DK2008/050129.

Stabilization of an IgG4 can be achieved by modification of the CH3 region or by modification of the hinge region.

Thus, in one embodiment, said heavy chain comprises a human IgG4 constant region having a residue selected from the group consisting of: Lys, Ala, Thr, Met, Leu and Trp at the position corresponding to 289 in SEQ ID NO:1 and/or a residue selected from the group consisting of: Ala, Val, Gly, Ile and Leu at the position corresponding to 285 in SEQ ID NO:1, and wherein said antibody optionally comprises one or more further substitutions, deletions and/or insertions. Preferably, said antibody comprises a Lys, Met or Leu residue at the position corresponding to 289, or said antibody comprises an Ala or Leu residue at the position corresponding to 285. In another embodiment, said stabilized IgG4 antibody comprises an Asp in the position corresponding to position 229 in SEQ ID NO:1 and/or a Lys in the position corresponding to in position 231 and/or a Thr in the position corresponding to position 237 and/or a Thr or Asp in the position corresponding to position 244 and/or a Thr, Gln or Glu in the position corresponding to position 250 and/or a Phe or Val at the position corresponding to position 291 in SEQ ID NO:1. The antibody optionally comprises one or more further substitutions, deletions and/or insertions in the constant region as set forth in SEQ ID NO:1.

In another embodiment, said IgG4 antibody has been modified to comprise a Cys-Pro-Pro-Cys sequence in the hinge region.

### Target Molecules

In one embodiment all antibodies of the mixture produced by the method of the invention are directed against the same target (i.e. the same antigen). Preferably, the antibodies of the mixture do not compete for binding to said target.

In another embodiment two or more antibodies in the mixture are directed against different targets.

In one embodiment of the invention, the resulting mixture is a mixture of monovalent and bivalent antibodies, wherein the bivalent antibodies are directed against targets for which immunocompetence is desired, (e.g. targets on the surface of tumor cells, where killing through effector mechanisms is desired) and the monovalent antibodies are directed against immune regulatory molecules, e.g. immune inhibitory molecules, thereby inhibiting them from binding to their receptors or blocking complement defense molecules.

In one embodiment, a mixture of monovalent antibodies of the invention specifically binds a cell surface receptor that is activated upon receptor dimerization. Monovalent antibodies may often be useful in the treatment of diseases or disorders where receptor activation is undesirable, since the antibody molecules of the inventions due to their monovalent nature are unable to induce such dimerization and thereby such activation. Without being limited to specific receptors, examples of such receptors could be erb-B1, erb-B2, erb-B3, erb-B4 and members of the ephrins and ephrin receptors such as ephrin-A1 through A8 and eph-B1 through eph-B6.

In another embodiment, a mixture of monovalent antibodies produced by the method of the invention, when bound to a target molecule, inhibits target molecule multimerization (such as dimerization). Again, such monovalent antibodies may be useful in the treatment of diseases or disorders where multimerization of the target antigen is undesirable, since the antibody molecules of the invention due to their monovalent nature are unable to induce such multimerization. In the case of soluble antigens, multimerization may form undesirable immune complexes. Without being limited to specific targets, examples of such targets could be ligands of Toll-like receptors such as TLR-3 and TLR-9, or angiopoietin-1, or angiopoietin-2, or TNF receptor family members such as CD30, CD40 and CD95.

As previously described, in certain pathological conditions, it is necessary and/or desirable to utilize monovalent antibodies. The monovalent antibodies in the mixture generated by the method of the invention are deficient in the activation of effector functions, such as ADCC and CDC.

In one embodiment of the invention, a mixture of monovalent and bivalent antibodies is produced by the method of the invention. Thus, the resulting mixture will typically (unless e.g. the bivalent antibody is of the IgG4 isotype) contain both bivalent antibodies capable of activating effector functions, such as ADCC and CDC and monovalent antibodies not capable of activating these functions.

The specific choice and utility of a mixture of antibodies of the invention for a particular purpose is dependent on the specific target of the antibody. The selection of targets for which a mixture of antibodies of the invention is useful for therapeutics and prophylactics may be based on the therapeutic value of administering an antibody specific for the target, or specific for a given epitope on the target. Such considerations are within the skills of the person skilled in the art.

One embodiment of the invention involves antibody mixtures useful for the treatment of solid tumors such as breast, gastro-intestinal, lung, ovarian, prostate tumors, etc. The cancer targets mentioned below can be targeted by a mixture of monovalent antibodies e.g. directed against different epitopes on the same target (wherein the antibodies of the mixture do not compete for binding to said target) or against different targets or by a mixture of monovalent and bivalent antibodies that bind different targets. In an embodiment of the invention the cancer targets are selected from cMet, EGFr, Her2 or HERV-envelop protein. In an embodiment a mixture of monovalent antibodies directed against periostin, Bigh3 and SPARC can be used in the treatment of solid tumors.

An embodiment of the invention involves antibody mixtures useful for the treatment of lymphoma. In one embodiment the targets are CD20, CD38, BCR, CD19, CD79, CD37. In one embodiment lymphoma is B-CLL. In an embodiment of the above the mixture of antibodies produced by the present invention is directed against a combination of CD38 and RANKL.

Another embodiment of the invention involves antibody mixtures useful for the treatment of multiple myeloma. This indication can be targeted by monovalent antibodies or a mixture of monovalent and bivalent antibodies directed against CD38 and CXCR4.

Another embodiment of the invention involves antibody mixtures useful for the treatment of CLL. This indication can be targeted by monovalent antibodies or a mixture of monovalent and bivalent antibodies against CD20 and CXCR4. Alternatively a mixture of monovalent antibodies or a mixture of monovalent and bivalent antibodies can be targeted against CD20 and CXCR4 and/or CCR7 and/or CXCR5.

A further embodiment of the invention involves antibody mixtures useful for the treatment of glioma. Such treatment can be targeted by a mixture of antibodies according to the present invention directed against EGFrwt, EGFrvIII and MRP3.

An even further embodiment of the invention involves antibody mixtures useful for the treatment of angiogenesis. The angiogenesis targets mentioned below can be targeted by a mixture of monovalent antibodies. The antibodies can be directed against different epitopes on the same target, wherein the antibodies of the mixture do not compete for binding to said target) or against different targets. In one embodiment these targets are Fibroblast growth factors (FGFs), Granulocyte colony-stimulating factor (G-CSF), Hepatocyte growth factor (HGF), Interleukin 8, Platelet-derived endothelial cell growth factor (PD-ECGF), Platelet-derived growth factor-BB (PDGF-BB), Pleiotrophin (PTN), Progranulin, Proliferin, Transforming growth factor-alpha (TGF-alpha), Transforming growth factor-beta (TGF-beta), Tumor necrosis factor-alpha (TNF-alpha), Vascular endothelial growth factor (VEGF), VEGF-C, VEGF-D and the like.

In another embodiment the targets include angiogenesis inhibitors, including but not limited to, Angiostatin (plasminogen fragment), Anti-angiogenic antithrombin III, Endostatin (collagen XVIII fragment), Fibronectin fragment, Gro-beta, Heparinases, Interferon alpha/beta/gamma, Interferon inducible protein (IP-10), Interleukin-12, Metalloproteinase inhibitors (TIMPs), Plasminogen activator inhibitor, and Thrombospondin-1 (TSP-1).

In a further embodiment of the invention a mixture of antibodies directed against a combination of VEGF or beta2GP1 in combination with lactadherin could be used in the treatment of undesired angiogenesis.

In an embodiment the above treatment of cancer by administration of a mixture of antibodies produced by the present invention can be combined with the anti-angiogenesis targets in the same manner as described above.

In an embodiment the above anti-angiogenesis targets can be combined with the anti-proteases targets in the same manner as described above.

In an embodiment the above treatment of cancer by administration of a mixture of antibodies produced by the present invention can be combined with antibodies against complement defense molecules such as CD55, CD59, and CD46 in the same manner as described above.

In an embodiment the above treatment of cancer by administration of a mixture of antibodies produced by the present invention can be combined with a mixture of monovalent antibodies modulating and activating the immune system, for example but not limited to CD80, CD86, CD200 or CD200R pathway, FcyRI (CD64), FcyRIIa (CD32a), FcyRIIc (CD32c) and FcyRIII (CD16) and/or inhibiting down modulating receptors including but not limited to KIR, FcyRIIb (CD32b) resulting in an immunostimulatory effect.

In an embodiment the mixture of antibodies produced according to the present invention can be used in the treatment of inflammatory diseases such as arthritis by targeting CD20 and RANKL. Another inflammatory disease like IBD can be targeted by providing a mixture of antibodies produced by the present invention against the targets CH3L1 and chitine binding protein.

In an embodiment the mixture of antibodies produced according to the present invention can be used in the treatment of Alzheimer's disease by targeting tau protein, APP differential structures of amyloid beta like monomeric structures combined with oligomeric structures and fibril structures.

In another embodiment infectious diseases are treated by the mixtures of antibodies according to the present invention. The infectious diseases may be of bacterial, viral, fungal, protozoa or parasite origin and the mixtures of antibodies as produced by the present invention may be directed against targets suitable for treatment of the diseases. The antibodies can be directed against different epitopes on the same target (wherein the antibodies of the mixture do not compete for binding to said target) or against different targets.

Infectious diseases might be caused by bacteria like, but not limited to, Bacillus antracis, Borrelia burgdorferi, Campylobacter jejuni, Chlamydia trachomatis, Clostridium botulinum, Clostridium tetani, Diptheria, E. coli, Legionella pneumofila, Helicobacter pylori, Mycobacterium tuberculosis, Mycobacterium bovis, Mycobacterium africanum, Mycobacterium leprae, Mycobacterium rikettsia, Mycoplasma neisseria, Neisseria meningitidis, Pertussis, Pseudomonas aeruginosa, Staphylococcus aureus, Staphylococcus epidermidis, Streptococcus and Yersinia pestis.

In an embodiment of the present invention tetanus and colitis resulting from clostridium toxins can be treated by providing a mixture of antibodies produced according to the present invention wherein the antibodies are targeted against specific antigens on the toxins.

Infectious diseases might also be caused by viruses like, but not limited to, adenovirus, cytomegalovirus, Epstein-Barr virus, hepatitis A, hepatitis B, hepatitis C, hepatitis D, hepatitis E, hepatitis F, hepatitis G, herpes simplex type I, herpes simplex type II, human immunodeficiency virus (HIV or HIV-1), human T-cell lymphotropic virus III, human papilloma virus (HPV), influenza virus type A, influenza virus type B, meningitis (viral), measles, papova virus, polio virus, respiratory syncytial virus, rhinovirus, rotavirus, rubella virus, SARS virus, and smallpox.

Infectious diseases might also be caused by fungi like, but not limited to, Aspergillus, Candida, cocci, and histoplasmosis.

Infectious diseases might also be caused by protozoa and parasites like, but not limited to, Chlamydia, Entamoeba histolytica, leishmania, Plasmodia (falciparum, vivax and malariae), rickettsia, and trypanosome.

In an embodiment malaria can be target by a mixture of antibodies produced according to the present invention by targeting a combination of AMA-1, MSP and GLURP.

In another embodiment the above treatment of infectious disease can be combined with a mixture of monovalent antibodies modulating and activating the immune system for example but not limited to CD200 or CD200R pathway, FcyRI (CD64), FcyRIIa (CD32a), FcyRIIc (CD32c), FcyRIII (CD16), and OX40 (CD134) and/or inhibiting down modulating receptors including but not limited to KIR, FcyRIIb (CD32b) resulting in an immunostimulatory effect.

In an embodiment HIV is treated with a combination of monovalent antibodies directed against two or more of: CD4, CCR5, CXCR4 and LFA-1.

In a further embodiment of the invention the diseases to be treated are inflammatory diseases like, but not limited to, acute respiratory distress syndrome (ARDS), arthritis (e.g., acute septic arthritis, psoriatic arthritis and rheumatoid arthritis including active rheumatoid arthritis and juvenile rheumatoid arthritis), asthma, Chron's disease, COPD, encephalitis, glomerulonephritis, Graves disease, inflammatory bowel disease, multiple sclerosis, myasthenia gravis, primary biniary cirrhosis, pemphigus, pemphigoid, septic shock, Sjögren syndrome, thrombotic thrombocytopenic purpura, type I diabetes mellitus, ulcerative colitis, transplant rejection.

The mixtures of antibodies of the present invention may also be combined with one or more additional therapeutic agents, such as anti-inflammatory agents, DMARDs (disease-modifying anti-rheumatic drugs), immunosuppressive agents, chemotherapeutics, and anti-psoriasis agents.

The expressed antibodies in the mixture of the present invention also encompass “derivatives” of monovalent antibodies, wherein one or more of the amino acid residues have been derivatised, for instance by acylation or glycosylation, without significantly affecting or altering the binding characteristics of the antibody containing the amino acid sequences. In the context of the present invention, a derivative of a monovalent antibody may for instance be a monovalent antibody, in which one or more of the amino acid residues of the monovalent antibody have been chemically modified (for instance by alkylation, acylation, ester formation, or amide formation) or associated with one or more non-amino acid organic and/or inorganic atomic or molecular substituents (for instance a polyethylene glycol (PEG) group, a lipophilic substituent (which optionally may be linked to the amino acid sequence of the peptide by a spacer residue or group such as β-alanine, γ-aminobutyric acid (GABA), L/D-glutamic acid, succinic acid, and the like), a fluorophore, biotin, a radionuclide, etc.) and may also or alternatively comprise non-essential, non-naturally occurring, and/or non-L amino acid residues, unless otherwise stated or contradicted by context. Non-limiting examples of such amino acid residues include for instance 2-aminoadipic acid, 3-aminoadipic acid, β-alanine, β-aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxylysine, allohydroxylysine, 3-hydroxyproline, 4-hydroxyproline, isodesmosine, alloisoleucine, N-methylglycine, N-methylisoleucine, 6-N-methyllysine, N-methylvaline, norvaline, norleucine, ornithine, and statine halogenated amino acids.

The antibodies expressed in the present invention may also be fused to other peptides, proteins or therapeutically active compounds.

The in vivo half-life of the antibodies may for instance be improved by modifying the salvage receptor epitope of the Ig constant domain or an Ig-like constant domain such that the molecule does not comprise an intact CH2 domain or an intact Ig Fc region, cf. U.S. Pat. No. 6,121,022 and U.S. Pat. No. 6,194,551. The in vivo half-life may be furthermore increased by making mutations in the Fc region, for instance by substituting threonine for leucine at the position corresponding to position 252 of an intact antibody molecule, threonine for serine at the position corresponding to position 254 of an intact antibody molecule, or threonine for phenylalanine at the position corresponding to position 256 of an intact antibody molecule, cf. U.S. Pat. No. 6,277,375.

In one embodiment, the antigen is a human protein molecule and the subject is a human subject. In one embodiment, the subject may be a non-human mammal expressing the antigen with which an antibody of the invention binds. Moreover, a mixture of monovalent antibodies of the invention may be administered to a non-human mammal expressing an antigen with which the immunoglobulin cross-reacts (for instance a primate, pig or mouse) for veterinary purposes or as an animal model of human disease. Regarding the latter, such animal models may be useful for evaluating the therapeutic efficacy of antibodies of the invention (for instance testing of dosages and time courses of administration).

Mixtures of antibodies of the invention may be used either alone or in combination with other compositions in a therapy. For instance, a mixture of antibodies of the invention may be co-administered with one or more other antibodies, such as antibodies produced according to the present invention, one or more chemotherapeutic agent(s) (including cocktails of chemotherapeutic agents), one or more other cytotoxic agent(s), one or more anti-angiogenic agent(s), one or more cytokines, one or more growth inhibitory agent(s), one or more anti-inflammatory agent(s), one or more disease modifying antirheumatic drug(s) (DMARD), or one or more immunosuppressive agent(s), depending on the disease or condition to be treated. Where a mixture of antibodies of the invention inhibits tumor growth, it may be particularly desirable to combine it with one or more other therapeutic agent(s) which also inhibits tumor growth. Alternatively, or additionally, the patient may receive combined radiation therapy (for instance external beam irradiation or therapy with a radioactive labeled agent, such as an antibody). Such combined therapies noted above include combined administration (where the two or more agents are included in the same or separate formulations), and separate administration, in which case, administration of the antibody of the invention may occur prior to, and/or following, administration of the adjunct therapy or therapies.

A mixture of antibodies of the invention may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. In one embodiment, the mixture of monovalent antibodies may be formulated with one or more agents currently used to prevent or treat the disorder in question. The effective amount of such other agents depends on the amount of antibodies of the invention present in the formulation, the type of disorder or treatment, and other factors discussed above.

The mixtures of antibodies of the invention (and adjunct therapeutic agent) may be administered by any suitable means, including parenteral, such as intravenous or subcutaneous administration. In addition, the mixture of antibodies may be suitably administered by pulse infusion, particularly with declining doses of the mixture of antibodies.

For the prevention or treatment of disease, the appropriate dosage of a mixture of antibodies of the invention (when used alone or in combination with other agents such as chemotherapeutic agents) will depend on the type of disease to be treated, the type of antibody, the severity and course of the disease, whether the mixture of antibodies is administered for preventive, therapeutic or diagnostic purposes, previous therapy, the patient's clinical history and response to the antibody, and the discretion of the attending physician. The mixture of antibodies may be suitably administered to the patient at one time or over a series of treatments.

Also within the scope of the present invention are kits comprising pharmaceutical compositions of the invention comprising one or more antibodies of the invention and instructions for use. The kit may further comprise one or more additional agents, such as an immunosuppressive reagent, a cytotoxic agent or a radiotoxic agent, depending on the disease or disorder to be treated, or one or more additional antibodies of the invention (for instance a mixture of antibodies having a complementary activity).

In one embodiment, the present invention provides a pharmaceutical composition comprising a mixture of antibodies of the present invention. The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

Regardless of the route of administration selected, the antibodies of the present invention, which may be used in the form of a pharmaceutically acceptable salt or in a suitable hydrated form, and/or the pharmaceutical compositions of the present invention, are formulated into pharmaceutically acceptable dosage forms by conventional methods known to those of skill in the art.

As described above, in a further aspect, the invention relates to a recombinant antibody comprising a heavy chain and a light chain, wherein the light chain comprises the sequence as set forth in SEQ ID NO.8.

In one embodiment, the light chain further comprises a sequence selected from the group consisting of: SEQ ID NO:9, 10 and 11, such as a light chain comprising a sequence selected from the group consisting of: SEQ ID NO:12, 13 and 14.

In one embodiment, the antibody is a bispecific antibody. In a further embodiment, the antibody is a monovalent antibody. In an even further embodiment, the antibody is a polyclonal antibody, such as a polyclonal bivalent antibody or a polyclonal monovalent antibody.

In one embodiment, the antibody is for use as a medicament, e.g. for the treatment of cancer.

### EXAMPLES

### Example 1

### Expression of Two Monovalent Human Antibodies with a Common Light Chain in a Single Cell

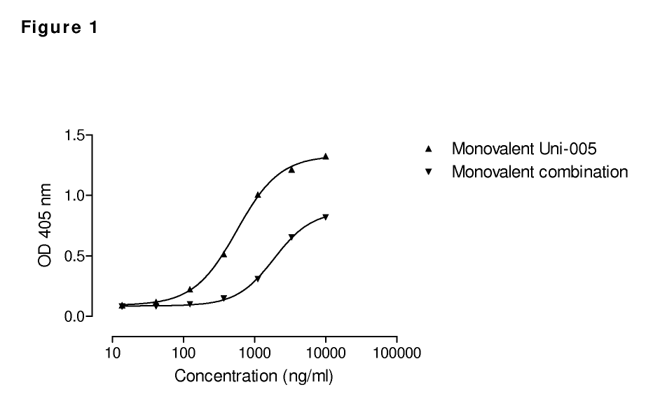

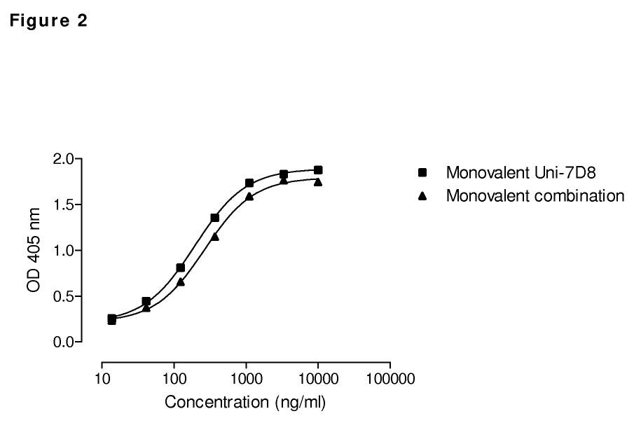

The expression vectors for the heavy chains (HC) of two antibodies, the anti-CD20 antibody 7D8 (WO2004035607) and the anti-CD38 antibody 005 (WO2006099875) were modified to change the isotype to IgG4 and to delete the sequence encoding the hinge region (the sequence coding for ESKYGPPCPSCP was deleted) (see also WO2007/059782). The resulting constructs were co-expressed with the light chain (LC) of 005 by transient co-expression in HEK-293F cells (Invitrogen, according to the recommendations of the manufacturer). Expression levels were measured by nephelometry and were in the normal range for expression in this system. The potential combination of two different monovalent antibodies in the supernatant was tested by ELISA to detect binding on soluble CD38 by ELISA as described in WO2006099875 and binding to an anti-idiotype antibody against 7D8 (described in Example 16 of WO2004035607). In FIGS. 1 and 2, it is shown that binding can be detected in the cell culture supernatant for both monovalent 7D8 and monovalent anti-CD38. Thus, two functional monovalent antibodies can be expressed in a single cell by using a common light chain.

### Example 2

### Production and Evaluation of Multiple Monovalent Antibodies with a Common Light Chain in a Single Cell Line

First the sequences encoding the VH regions of a panel of antibodies (specific for EGFr (clone LC1006-018, described in WO2009030239), c-Met and Her2 respectively) were cloned in a mammalian expression vector (pcDNA3.3, Invitrogen) containing the constant region of a hinge-modified, monovalent (hinge region E99-P110 deleted and containing the substitutions F273T and Y275E in the CH3 region (SEQ ID NO:2) as described in WO2008145140) human IgG4 antibody. To identify common light chains each of the HC vectors were transiently co-transfected in HEK-293F cells with a library of expression vectors encoding a single human LC kappa germline sequence. The library comprised a set of 200 germline kappa sequences (each of the 40 known functional V-Kappa segments combined with each of the 5 functional J-Kappa human germline sequences) that were obtained from the publicly available database VBASE (Tomlinson, I. M., Williams, S. C., Corbett, S. J., Cox, J. B. L., Winter, G., 1996. VBASE Sequence Directory. MRC Centre for Protein Engineering, Cambridge, UK (http://vbase.mrc-cpe.cam.ac.uk/)). To identify common light chains, the supernatants of all transient transfected cell cultures were collected 5 days after transfection, diluted 20 times and screened for the presence of functional antibodies by performing a binding ELISA using a recombinant purified soluble antigen target coated to the plate, as described below. The concentration of IgG in the supernatants was determined by an Octet Dip and Read™ assay (ForteBio) using an anti-human IgG Fc biosensor coated on the tip surface.

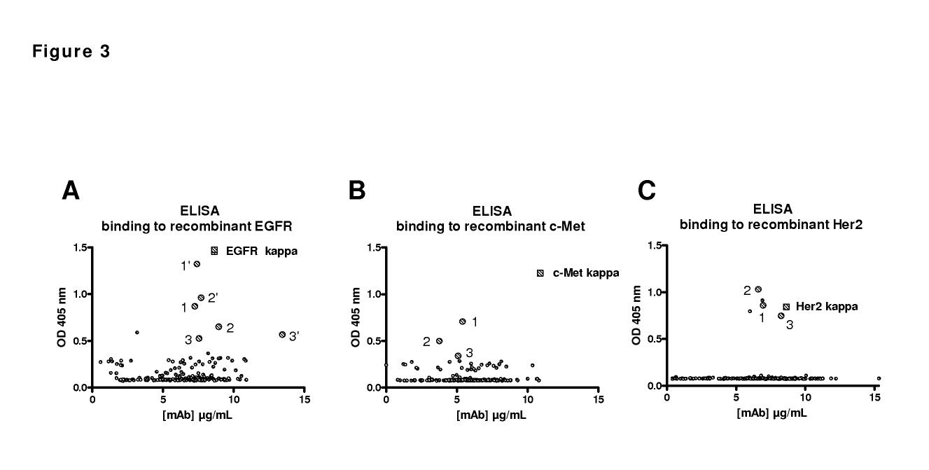

In FIG. 3 the results of the screening by binding ELISAs are shown. Three out of the 200 LC kappa germline sequences were identified (common light chain 1, 2 and 3) to form a functional antibody in combination with all three different hinge-modified (F273T, Y275E) heavy chains, each with a different antigen specificity. The identified common light chains were composed of V-segment VKVI-2-1-(1)-A14 (IGKV6D-41*01) [DVVMTQSPAFLSVTPGEKVTITCQASEGIGNYLYWYQQKPDQAPKLLIKYASQSISG VPSRFSGSGSGTDFTFTISSLEAEDAATYYCQQGNKHP (SEQ ID NO:8)] combined with either JK-segment JK1 (IGKJ1*01) [WTFGQGTKVEIK (SEQ ID NO:9)] (common light chain 1), JK2 (IGKJ2*01) [YTFGQGTKLEIK (SEQ ID NO:10)] (common light chain 2) or JK3 (IGKJ3*01) [FTFGPGTKVDIK (SEQ ID NO:11)] (common light chain 3).

Thus, the identified common light chain sequences were as follows:

Sequence common light chain 1:
(SEQ ID NO: 12)
DVVMTQSPAFLSVTPGEKVTITCQASEGIGNYLYWYQQKPDQAPKLLIK
YASQSISGVPSRFSGSGSGTDFTFTISSLEAEDAATYYCQQGNKHPWTF
GQGTKVEIK
Sequence common light chain 2:
(SEQ ID NO: 13)
DVVMTQSPAFLSVTPGEKVTITCQASEGIGNYLYWYQQKPDQAPKLLIK
YASQSISGVPSRFSGSGSGTDFTFTISSLEAEDAATYYCQQGNKHPYTF
GQGTKLEIK
Sequence common light chain 3:
(SEQ ID NO: 14)
DVVMTQSPAFLSVTPGEKVTITCQASEGIGNYLYWYQQKPDQAPKLLIK
YASQSISGVPSRFSGSGSGTDFTFTISSLEAEDAATYYCQQGNKHPFTF
GPGTKVDIK

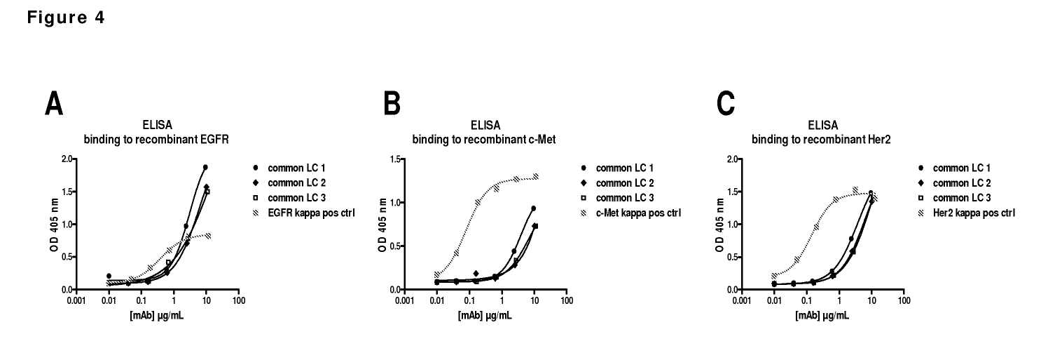

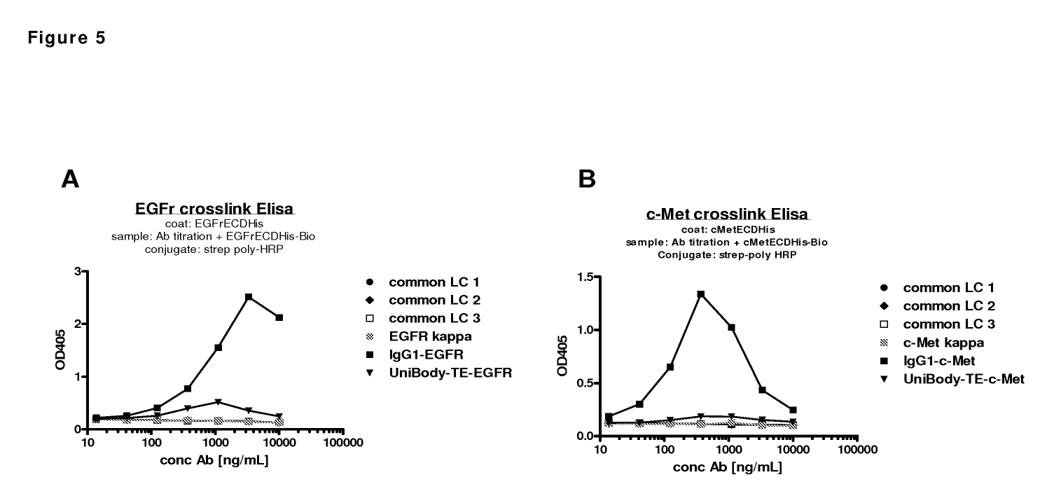

These results were confirmed by co-expressing the 3 heavy chains together with each of the identified common light chains in one cell. Expression of three different functional antibodies in a single cell was determined by testing the supernatants for binding to all three recombinant antigens in three individual ELISAs, as described below. In FIG. 4, the results of the three individual ELISAs are shown. Binding to each of the recombinant antigens used as coat is observed with the supernatant containing a mixture of three different functional monovalent antibodies expressed in one cell. To confirm that the antibodies were monovalent, a crosslink ELISA was performed. In this assay, two versions of the target antigen were used. Recombinant soluble antigen was used as coat for the ELISA. Bivalent antibodies against the target (EGFr, c-Met or Her2) are then detected by addition of a biotinylated version of the antigen and a subsequent detection by streptavidin-HRP. FIG. 5 shows that in this ELISA, no signal was observed for the antibodies from the mixture, confirming the monovalency of at least the anti-EGFr and anti-c-Met material (monovalency of the anti-Her2 material was not tested).

Binding to Recombinant EGFr, c-Met and Her2 in ELISA

Recombinant soluble c-Met-Fc chimera (R&D systems), EGFrECDHis (His-tagged extracellular EGFr domain) and Her2ECDHis (His-tagged extracellular Her2 domain) were produced and coated to 96-well flat-bottom Microlon ELISA plates (Greiner, Frickenhausen, Germany; product# 655092) by incubating overnight at 4° C. Wells were emptied and blocked with PBSC (PBS supplemented with 2% chicken serum) at room temperature for 60 min. Plates are washed thrice with PBST using an EL404 Microplate Autowasher (Bio-Tek Instruments). 1:20 dilutions of supernatant in PBSTC (PBS supplemented with 2% chicken serum and 0.05% Tween-20). Wells were incubated at room temperature for 1 h while shaking at 300 rpm. Plates were washed thrice with PBSTC and wells were incubated with HRP-conjugated mouse-anti-human IgG Fc specific (CLB, The Netherlands; 1:10,000 diluted in PBSTC, 100 μl/well) at room temperature for 1 h. Plates were washed thrice with PBST. Wells were incubated with freshly prepared ABTS solution (ABTS: 2,2′-azino bis(3-ethylbenzthiazoline-6-sulfonic acid; tablets in ABTS buffer [Roche Diagnostics] to 1 mg/mL) in the dark at room temperature for 30 min. Absorbance was measured at 405 nm using an EL808 Ultra Microplate Reader with KC4™ software (Bio-Tek Instruments).

SEQUENCE LISTING

SEQ ID NO: 1: Amino acid sequence of the wild type constant domain
of the heavy chain (CH) of human IgG4.

| | |
|---|---|
| 1 | ASTKGPSVFP LAPCSRSTSE STAALGCLVK DYFPEPVTVS WNSGALTSGV |
| 51 | 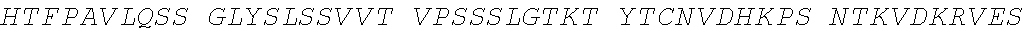 |
| 101 | 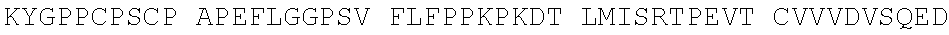 |
| 151 | PEVQFNWYVD GVEVHNAKTK PREEQFNSTY RVVSVLTVLH QDWLNGKEYK |
| 201 | CKVSNKGLPS SIEKTISKAK GQPREPQVYT LPPSQEEMTK NQVSLTCLVK |
| 251 | GFYPSDIAVE WESNGQPENN YKTTPPVLDS DGSFFLYSRL TVDKSRWQEG |
| 301 | NVFSCSVMHE ALHNHYTQKS LSLSLGK |

SEQ ID NO: 2: Amino acid sequence of the mutant constant domain of
the heavy chain (CH) of human IgG4, in which the hinge region is
deleted

| | |
|---|---|
| 1 | ASTKGPSVFP LAPCSRSTSE STAALGCLVK DYFPEPVTVS WNSGALTSGV |
| 51 | 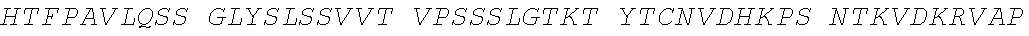 |
| 101 | 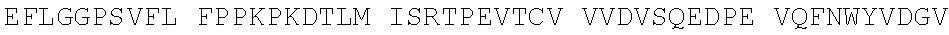 |
| 151 | EVHNAKTKPR EEQFNSTYRV VSVLTVLHQD WLNGKEYKCK VSNKGLPSSI |
| 201 | EKTISKAKGQ PREPQVYTLP PSQEEMTKNQ VSLTCLVKGF YPSDIAVEWE |
| 251 | SNGQPENNYK TTPPVLDSDG SFFLYSRLTV DKSRWQEGNV FSCSVMHEAL |
| 301 | HNHYTQKSLS LSLGK |

NO: 3: Amino acid sequence of the constant domain of the human
lambda light chain (CL) (accession number S25751)

| | |
|---|---|
| 1 | QPKAAPSVTL FPPSSEELQA NKATLVCLIS DFYPGAVTVA WKADSSPVKA |
| 51 | GVETTTPSKQ SNNKYAASSY LSLTPEQWKS HRSYSCQVTH EGSTVEKTVA |
| 101 | PTECS |

SEQ ID NO: 4: Amino acid sequence of the constant domain of the
human kappa light chain (CL) (accession number P01834).

| | |
|---|---|
| 1 | TVAAPSVFIF PPSDEQLKSG TASVVCLLNN FYPREAKVQW KVDNALQSGN |
| 51 | SQESVTEQDS KDSTYSLSST LTLSKADYEK HKVYACEVTH QGLSSPVTKS |
| 101 | FNRGEC |

SEQ ID NO: 5: Amino acid sequence of the constant domain of the
heavy chain (CH)of human IgG1 (accession number P01857)

| | |
|---|---|
| 1 | ASTKGPSVFP LAPSSKSTSG GTAALGCLVK DYFPEPVTVS WNSGALTSGV |
| 51 | 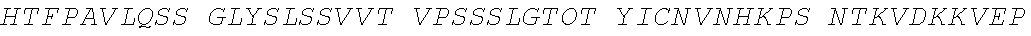 |
| 101 | 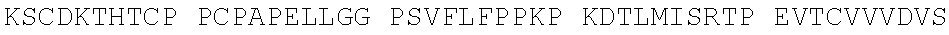 |
| 151 | HEDPEVKFNW YVDGVEVHNA KTKPREEQYN STYRVVSVLT VLHQDWLNGK |
| 201 | EYKCKVSNKA LPAPIEKTIS KAKGQPREPQ VYTLPPSRDE MTKNQVSLTC |
| 251 | LVKGFYPSDI AVEWESNGQP ENNYKTTPPV LDSDGSFFLY SKLTVDKSRW |
| 301 | QQGNVFSCSV MHEALHNHYT QKSLSLSPGK |

SEQ ID NO: 6: Amino acid sequence of the constant domain of the
heavy chain (CH) of human IgG2 (accession number P01859)

| | |
|---|---|
| 1 | ASTKGPSVFP LAPCSRSTSE STAALGCLVK DYFPEPVTVS WNSGALTSGV |
| 51 | 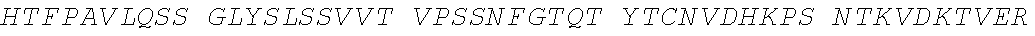 |
| 101 | 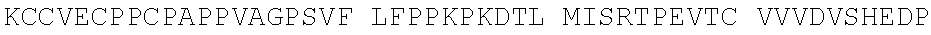 |
| 151 | EVQFNWYVDG VEVHNAKTKP REEQFNSTFR VVSVLTVVHQ DWLNGKEYKC |
| 201 | KVSNKGLPAP IEKTISKTKG QPREPQVYTL PPSREEMTKN QVSLTCLVKG |
| 251 | FYPSDIAVEW ESNGQPENNY KTTPPMLDSD GSFFLYSKLT VDKSRWQQGN |
| 301 | VFSCSVMHEA LHNHYTQKSL SLSPGK |

SEQ ID NO: 7: Amino acid sequence of the constant domain of the
heavy chain (CH)of human IgG3

| | |
|---|---|
| 1 | ASTKGPSVFP LAPCSRSTSG GTAALGCLVK DYFPEPVTVS WNSGALTSGV |
| 51 | 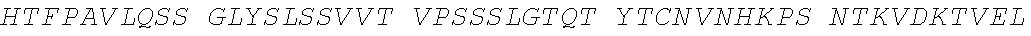 |
| 101 | 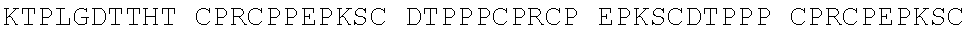 |
| 151 | 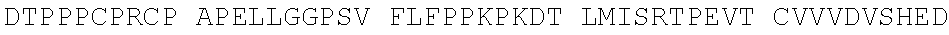 |
| 201 | PEVQFKWYVD GVEVHNAKTK PREEQYNSTF RVVSVLTVLH QDWLNGKEYK |
| 251 | CKVSNKALPA PIEKTISKTK GQPREPQVYT LPPSREEMTK NQVSLTCLVK |
| 301 | GFYPSDIAVE WESSGQPENN YNTTPPMLDS DGSFFLYSKL TVDKSRWQQG |
| 351 | NIFSCSVMHE ALHNRFTQKS LSLSPGK |

SEQ ID NO: 8: Amino acid sequence of V-segment VKVI-2-1-(1)-A14
(IGKV6D-41*01):

| | |
|---|---|
| 1 | DVVMTQSPAF LSVTPGEKVT ITCQASEGIG NYLYWYQQKP DQAPKLLIKY |
| 51 | ASQSISGVPS RFSGSGSGTD FTFTISSLEA EDAATYYCQQ GNKHP |

SEQ ID NO: 9: Amino acid sequence of JK-segment JK1 (IGKJ1*01)

| | |
|---|---|
| 1 | WTFGQGTKVE IK |

SEQ ID NO: 10: Amino acid sequence of JK-segment JK2 (IGKJ2*01)

| | |
|---|---|
| 1 | YTFGQGTKLE IK |

SEQ ID NO: 11: Amino acid sequence of JK-segment JK3 (IGKJ3*01)

| | |
|---|---|
| 1 | FTFGPGTKVD IK. |

SEQ ID NO: 12: Amino acid sequence of common light chain 1:

| | |
|---|---|
| 1 | DVVMTQSPAF LSVTPGEKVT ITCQASEGIG NYLYWYQQKPDQAPKLLIKY |
| 51 | ASQSISGVPS RFSGSGSGTD FTFTISSLEA EDAATYYCQQGNKHPWTFGQ |
| 101 | GTKVEIK |

SEQ ID NO: 13: Amino acid sequence of common light chain 2:

| | |
|---|---|
| 1 | DVVMTQSPAF LSVTPGEKVT ITCQASEGIG NYLYWYQQKPDQAPKLLIKY |
| 51 | ASQSISGVPS RFSGSGSGTD FTFTISSLEA EDAATYYCQQGNKHPYTFGQ |
| 101 | GTKLEIK |

SEQ ID NO: 14: Amino acid sequence of common light chain 3:

| | |
|---|---|
| 1 | DVVMTQSPAF LSVTPGEKVT ITCQASEGIG NYLYWYQQKPDQAPKLLIKY |
| 51 | ASQSISGVPS RFSGSGSGTD FTFTISSLEA EDAATYYCQQGNKHPFTFGP |
| 101 | GTKVDIK |

1. A method for producing a mixture comprising two or more different antibodies in a single recombinant host cell, comprising expressing in said host cell: a) at least one nucleic acid construct encoding a common light chain, and b) two or more nucleic acid constructs encoding a heavy chain, said two or more nucleic acid constructs comprising b1) two or more nucleic acid constructs encoding two or more different first heavy chains, wherein the amino acid sequence of each of the constant regions of the first heavy chains has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being, or b2) at least one nucleic acid construct encoding a first heavy chain, wherein the amino acid sequence of the constant region has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being, and at least one nucleic acid construct encoding a second heavy chain which is capable of forming disulphide bonds or covalent or stable non-covalent inter-heavy chain bonds with an identical CH region in the presence of IVIG or when administered to a mammal or human being, wherein each of the heavy chains is capable of pairing with the light chain to form a functional antibody.

2. The method according to claim 1, wherein the method comprises expressing in said host cell: two or more nucleic acid constructs encoding two or more different first heavy chains, wherein the amino acid sequence of each of the constant regions of the first heavy chains has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being.

3. The method according to claim 1, wherein the method comprises expressing in said host cell:
at least one nucleic acid construct encoding a first heavy chain, wherein the amino acid sequence of the constant region has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being, and
at least one nucleic acid construct encoding a second heavy chain which is capable of forming disulphide bonds or covalent or stable non-covalent inter-heavy chain bonds with an identical CH region in the presence of IVIG or when administered to a mammal or human being.

4. A method for producing a mixture comprising two or more different antibodies in a single recombinant host cell, comprising expressing in said host cell a) at least one nucleic acid construct encoding a common light chain, and b) two or more nucleic acid constructs encoding a heavy chain, said two or more nucleic acid constructs comprising b1) two or more nucleic acid constructs encoding two or more different first IgG4 heavy chains, wherein the amino acid sequence of each of the constant regions of the first IgG4 heavy chains has been modified so that the hinge region does not contain any amino acid residues which are capable of forming disulphide bonds or covalent or stable non-covalent inter-heavy chain bonds with an identical CH region in the presence of IVIG or when administered to a mammal or human being, or b2) at least one nucleic acid construct encoding a first IgG4 heavy chain, wherein the amino acid sequence of the constant region has been modified so that the hinge region does not contain any amino acid residues which are capable of forming disulphide bonds or covalent or stable non-covalent inter-heavy chain bonds with an identical CH region in the presence of IVIG or when administered to a mammal or human being, and at least one nucleic acid construct encoding a second heavy chain which is capable of forming disulphide bonds or covalent or stable non-covalent inter-heavy chain bonds with an identical CH region in the presence of IVIG or when administered to a mammal or human being, with the proviso that the second heavy chain is not a wild type IgG4 heavy chain, wherein each of the heavy chains is capable of pairing with the light chain to form a functional antibody.

5. The method according to claim 1, wherein said at least one nucleic acid construct encoding a first heavy chain comprises a sequence encoding a CH3 region as set forth in SEQ ID NO: 5, but wherein the CH3 region has been modified so that one or more of the following amino acid substitutions have been made: Arg (R) in position 238 has been replaced by Gln (Q); Asp (D) in position 239 has been replaced by Glu (E); Thr (T) in position 249 has been replaced by Ala (A); Leu (L) in position 251 has been replaced by Ala (A); Leu (L) in position 251 has been replaced by VaI (V); Phe (F) in position 288 has been replaced by Ala (A); Phe (F) in position 288 has been replaced by Leu (L); Tyr (Y) in position 290 has been replaced by Ala (A); Lys (K) in position 292 has been replaced by Arg (R); Lys (K) in position 292 has been replaced by Ala (A); Gln (Q) in position 302 has been replaced by Glu (E); and Pro (P) in position 328 has been replaced by Leu (L).

6. The method according to claim 5, wherein Lys (K) in position 292 has been replaced by Arg (R).

7. The method according to claim 5, wherein said at least one nucleic acid construct encoding a first heavy chain further comprises a sequence encoding the CH 1 and/or CH2 regions as set forth in SEQ ID NO: 5.

8. The method according to claim 1, wherein the constant region of the light chain has been modified so that it does not contain any amino acids capable of forming disulfide bonds or other covalent bonds with an identical constant region in the presence of IVIG or when administered to a mammal or human being.

9. The method according to claim 8, wherein said at least one nucleic acid construct encoding a common light chain comprises a sequence encoding the kappa CL region having the amino acid sequence as set forth in SEQ ID NO: 4, but wherein the sequence has been modified so that the terminal cysteine residue in position 106 has been replaced with another amino acid residue or has been deleted.

10. The method according to claim 8, wherein said at least one nucleic acid construct encoding a common light chain comprises a sequence encoding the lambda CL region having the amino acid sequence as set forth in SEQ ID NO: 3, but wherein the sequence has been modified so that the cysteine residue in position 104 has been replaced with another amino acid residue or has been deleted.

11. The method according to claim 1, wherein the constant region of the heavy chain has been modified so that it contains a residue that is capable of a forming a disulfide bond or other covalent bond with the light chain.

12. The method according to claim 1, wherein said at least one nucleic acid construct encoding a first heavy chain comprises a sequence encoding a CH1 region as set forth in SEQ ID NO: 5, but wherein the CH1 region has been modified so that Ser (S) in position 14 has been replaced by a cysteine residue.

13. The method according to claim 1, wherein said at least one nucleic acid construct encoding a first heavy chain comprises a sequence encoding a CH3 region as set forth in SEQ ID NO: 6, but wherein the CH3 region has been modified so that one or more of the of the following amino acid substitutions have been made: Arg (R) in position 234 has been replaced by Gln (Q); Thr (T) in position 245 has been replaced by Ala (A); Leu (L) in position 247 has been replaced by Ala (A); Leu (L) in position 247 has been replaced by VaI (V); Met (M) in position 276 has been replaced by VaI (V); Phe (F) in position 284 has been replaced by Ala (A); Phe (F) in position 284 has been replaced by Leu (L); Tyr (Y) in position 286 has been replaced by Ala (A); Lys (K) in position 288 has been replaced by Arg (R); Lys (K) in position 288 has been replaced by Ala (A); Gln (Q) in position 298 has been replaced by Glu (E); and Pro (P) in position 324 has been replaced by Leu (L).

14. The method according to claim 13, wherein Lys (K) in position 288 has been replaced by Arg (R).

15. The method according to claim 1, wherein said at least one nucleic acid construct encoding a first heavy chain further comprises a sequence encoding the CH 1 and/or CH2 regions as set forth in SEQ ID NO: 6.

16. The method according to claim 1, wherein said at least one nucleic acid construct encoding a first heavy chain comprises a sequence encoding a CH3 region as set forth in SEQ ID NO: 7, but wherein the CH3 region has been modified so that one or more of the following amino acid substitutions have been made: Arg (R) in position 285 has been replaced by Gln (Q); Thr (T) in position 296 has been replaced by Ala (A); Leu (L) in position 298 has been replaced by Ala (A); Leu (L) in position 298 has been replaced by VaI (V); Ser (S) in position 314 has been replaced by Asn (N); Asn (N) in position 322 has been replaced by Lys (K); Met (M) in position 327 has been replaced by VaI (V); Phe (F) in position 335 has been replaced by Ala (A); Phe (F) in position 335 has been replaced by Leu (L); Tyr (Y) in position 337 has been replaced by Ala (A); Lys (K) in position 339 has been replaced by Arg (R); Lys (K) in position 339 has been replaced by Ala (A); Gln (Q) in position 349 has been replaced by Glu (E); He (I) in position 352 has been replaced by VaI (V); Arg (R) in position 365 has been replaced by His (H); Phe (F) in position 366 has been replaced by Tyr (Y); and Pro (P) in position 375 has been replaced by Leu (L).

17. The method according to claim 16, wherein Lys (K) in position 339 has been replaced by Arg (R).

18. The method according to claim 1, wherein said at least one nucleic acid construct encoding a first heavy chain further comprises a sequence encoding the CH 1 and/or CH2 regions as set forth in SEQ ID NO: 7.

19. The method according to claim 1, wherein said at least one nucleic acid construct encoding a first heavy chain comprises a sequence encoding a CH3 region as set forth in SEQ ID NO: 2, but wherein the CH3 region has been modified so that one or more of the following amino acid substitutions have been made: Thr (T) in position 234 has been replaced by Ala (A); Leu (L) in position 236 has been replaced by Ala (A); Leu (L) in position 236 has been replaced by VaI (V); Phe (F) in position 273 has been replaced by Ala (A); Phe (F) in position 273 has been replaced by Leu (L); Tyr (Y) in position 275 has been replaced by Ala (A).

20. The method according to claim 1, wherein said at least one nucleic acid construct encoding a first heavy chain comprises a sequence encoding a CH3 region as set forth in SEQ ID NO: 2.

21. The method according to claim 20, but wherein Glu (E) in position 225 has been replaced by Ala (A).

22. The method according to claim 20, but wherein Thr (T) in position 234 has been replaced by Ala (A).

23. The method according to claim 20, but wherein Leu (L) in position 236 has been replaced by Ala (A).

24. The method according to claim 20, but wherein Leu (L) in position 236 has been replaced by VaI (V).

25. The method according to claim 20, but wherein Leu (L) in position 236 has been replaced by Glu (E).

26. The method according to claim 20, but wherein Leu (L) in position 236 has been replaced by Gly (G).

27. The method according to claim 20, but wherein Lys (K) in position 238 has been replaced by Ala (A).

28. The method according to claim 20, but wherein Asp (D) in position 267 has been replaced by Ala (A).

29. The method according to claim 20, but wherein Phe (F) in position 273 has been replaced by Ala (A).

30. The method according to claim 20, but wherein Phe (F) in position 273 has been replaced by Leu (L).

31. The method according to claim 20, but wherein Phe (F) in position 273 has been replaced by Asp (D) and/or Tyr (Y) in position 275 has been replaced by Glu (E).

32. The method according to claim 20, but wherein Phe (F) in position 273 has been replaced by Thr (T) and/or Tyr (Y) in position 275 has been replaced by Glu (E).

33. The method according to claim 20, but wherein Tyr (Y) in position 275 has been replaced by Ala (A).

34. The method according to claim 20, wherein said at least one nucleic acid construct encoding a first heavy chain further comprises a sequence encoding the CH2 region as set forth in SEQ ID NO: 2, but wherein Thr (T) in position 118 has been replaced by Gln (Q) and/or Met (M) in position 296 has been replaced by Leu (L).

35. The method according to claim 20, wherein said at least one nucleic acid construct encoding a first heavy chain further comprises a sequence encoding the CH2 region as set forth in SEQ ID NO: 2, but wherein one, two or all three of the following substitutions have been made: Met (M) in position 120 has been replaced by Tyr (Y); Ser (S) in position 122 has been replaced by Thr (T); and Thr (T) in position 124 has been replaced by Glu (E).

36. The method according to claim 20, wherein said at least one nucleic acid construct encoding a first heavy chain further comprises a sequence encoding the CH2 region as set forth in SEQ ID NO: 2, but wherein Asn (N) in position 302 has been replaced by Ala (A).

37. The method according to claim 20, wherein said at least one nucleic acid construct encoding a first heavy chain further comprises a sequence encoding the CH2 region as set forth in SEQ ID NO: 2, but wherein Asn (N) in position 302 has been replaced by Ala (A) and Thr (T) in position 175 has been replaced by Ala (A) and Glu (E) in position 248 has been replaced by Ala (A).

38. The method according to claim 1, wherein said at least one nucleic acid construct encoding a first heavy chain comprises a sequence encoding a CH region which has been modified such that all cysteine residues in the hinge region have been deleted or substituted with other amino acid residues.

39. The method according to claim 38, wherein the CH region has been modified such that the cysteine residues of the hinge region have been substituted with amino acid residues that have an uncharged polar side chain or a nonpolar side chain.

40. The method according to claim 1, wherein said at least one nucleic acid construct encoding a first heavy chain comprises a sequence encoding a IgG4 CH region, wherein the amino acids corresponding to amino acids 106 and 109 of the CH sequence of SEQ ID No: 1 have been deleted.

41. The method according to claim 1, wherein said at least one nucleic acid construct encoding a first heavy chain comprises a sequence encoding a IgG4 CH region, wherein one of the amino acid residues corresponding to amino acid residues 106 and 109 of the sequence of SEQ ID No: 1 has been substituted with an amino acid residue different from cysteine, and the other of the amino acid residues corresponding to amino acid residues 106 and 109 of the sequence of SEQ ID No: 1 has been deleted.

42. The method according to claim 1, wherein said at least one nucleic acid construct encoding a first heavy chain comprises a sequence encoding a IgG4 CH region, wherein at least the amino acid residues corresponding to amino acid residues 106 to 109 of the CH sequence of SEQ ID No: 1 have been deleted.

43. The method according to claim 1, wherein said at least one nucleic acid construct encoding a first heavy chain comprises a sequence encoding a IgG4 CH region, wherein at least the amino acid residues corresponding to amino acid residues 99 to 110 of the sequence of SEQ ID No: 1 have been deleted.

44. The method according to claim 1, wherein said at least one nucleic acid construct encoding a first heavy chain comprises a sequence encoding a CH region which, except for any mutations specified in any of the preceding claims, comprises the amino acid sequence of SEQ ID No: 2.

45. The method according to claim 1, wherein said at least one nucleic acid construct encoding a first heavy chain comprises a sequence encoding an IgG4 CH region, wherein the CH region has been modified such that the entire hinge region has been deleted.

46. The method according to claim 1, wherein said at least one nucleic acid construct encoding a first heavy chain comprises a sequence encoding a CH region which has been modified so that it does not comprise any acceptor sites for N-linked glycosylation.

47. The method according to claim 46, wherein the NST acceptor site for N-linked glycosylation in the CH2 region has been modified to a sequence selected from the group consisting of: GST, MST, CSE, DSE, DSP, ESP, GSP, HSE, NSE, PSP and SSE.

48. The method according to claim 1, wherein the second heavy chain is an IgG1, IgG2, IgG3 or stabilized IgG4 heavy chain or IgA, preferably an IgG1 heavy chain.

49. The method according to claim 1, wherein at least one, at least two, e.g. all of the antibodies in the mixture are human antibodies.

50. The method according to claim 1, wherein a mixture of three or more different antibodies, such as a mixture of four or more different antibodies, e.g. a mixture of five or more different antibodies is produced.

51. The method according to claim 1, wherein a mixture of less than twenty different antibodies is produced.

52. The method according to claim 1, wherein said host cell comprises more than one nucleic acid construct encoding a light chain, preferably wherein each of the heavy chains is capable of pairing with each of the light chain to form a functional antibody.

53. The method according to claim 1, wherein all antibodies of the mixture are directed against the same target.

54. The method according to claim 53, wherein the antibodies of the mixture do not compete for binding to said target.

55. The method according to claim 1, wherein two or more antibodies in the mixture are directed against different targets.

56. The method according to claim 1, wherein the method comprises culturing said host cell for at least 20 population doublings.

57. The method according to claim 1, wherein the method comprises the further step of harvesting the mixture from the cell culture.

58. The method according to claim 1, wherein the method comprises the further step of purifying the antibody mixture.

59. The method according to claim 1, wherein said host cell is a mammalian cell, such as a CHO cell.

60. The method according to claim 1, wherein the common light chain comprises the sequence as set forth in SEQ ID NO.8.

61. The antibody according to claim 60, wherein the common light chain further comprises a sequence selected from the group consisting of: SEQ ID NO:9, 10 and 11, such as a common light chain comprising a sequence selected from the group consisting of: SEQ ID NO: 12, 13 and 14.

62. The method according to claim 1 wherein said nucleic acid are stably integrated into the genome of the host cell.

63. A composition comprising a mixture of antibodies obtainable by the method of claim 1.

64. The composition according to claim 63 for use as a medicament.

65. A recombinant host cell suitable for use in the production of a mixture comprising two or more different antibodies, wherein said host cell comprises: a) at least one nucleic acid construct encoding a common light chain, and b) two or more nucleic acid constructs encoding a heavy chain, said two or more nucleic acid constructs comprising b1) two or more nucleic acid constructs encoding two or more different first heavy chains, wherein the amino acid sequence of each of the constant regions of the first heavy chains has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being, or b2) at least one nucleic acid construct encoding a first heavy chain, wherein the amino acid sequence of the constant region has been modified so that the hinge region and, as required by the immunoglobulin subtype, other regions of the CH region, such as the CH3 region, does not contain any amino acid residues which are capable of forming disulphide bonds or covalent or stable non-covalent inter-heavy chain bonds with an identical CH region in the presence of IVIG or when administered to a mammal or human being, and at least one nucleic acid construct encoding a second heavy chain which is capable of forming disulphide bonds or covalent or stable non-covalent inter-heavy chain bonds with an identical CH region in the presence of IVIG or when administered to a mammal or human being,

66. The host cell according to claim 65, wherein said host cell is a mammalian cell, such as a CHO cell.

67. A recombinant antibody comprising a heavy chain and a light chain, wherein the light chain comprises the sequence as set forth in SEQ ID NO.8.

68. The antibody according to claim 67, wherein the light chain further comprises a sequence selected from the group consisting of: SEQ ID NO:9, 10 and 11, such as a light chain comprising a sequence selected from the group consisting of: SEQ ID NO: 12, 13 and 14.

69. The antibody according to claim 67, wherein the antibody is a bispecific antibody.

70. The antibody according to claim 67, wherein the antibody is a monovalent antibody.

71. The antibody according to claim 67, wherein the antibody is a polyclonal antibody, such as a polyclonal bivalent antibody or a polyclonal monovalent antibody.

72. The antibody according to claim 67 for use as a medicament, such as a medicament for the treatment of cancer.
